(19) United States
(12) Patent Application Publication
(10) Pub. No.: US 2002/0177863 A1
(43) Pub. Date: Nov. 28, 2002

(54) Surface treated ligating clip

(76) Inventors: Stanley R. Mandel, Chapel Hill, NC (US); James R. Whitney, Morrisville, NC (US)

Correspondence Address: Michael G. Savage, BURNS, DOANE, SWECKER & MATHIS, L.L.P., P.O. Box 1404, Alexandria, VA, 22313-1404, US

(21) Appl. No.: 09/864,585

(22) Filed: May 24, 2001

(51) Int. Cl. A61B 17/08

(52) U.S. Cl. 606/158

(57) ABSTRACT

The invention provides ligating clips for surgical use which have antimicrobial or antibiotic properties. More particularly, the ligating clips have antimicrobial and/or antibiotic agents either in a coating or provided within the material from which the clip is manufactured. The invention also provides a method for inhibiting the growth of or killing microorganisms by coating a ligating clip or ligating clip applying instrument with an antimicrobial or antibiotic coating composition or incorporating an antimicrobial or antibiotic coating into the material from which the ligating clip or ligating clip applying instrument was made.

### FIELD OF THE INVENTION

The present invention relates to surgical ligating clips and clip appliers and more particularly to the use of antibiotic and/or antimicrobial coatings or additives for such devices.

### BACKGROUND OF THE INVENTION

Surgical clips like hemostatic clips and aneurysm clips are often used in surgery to ligate vessels or tissue bundles to stop the flow of blood. The clips may be left in place permanently or may be absorbable. Within a period of time the ligated end of the vessel will close, that is, hemostasis or occlusion will occur.

Metal clips having generally U or chevron shapes have been used for years. The most common metals are tantalum, titanium, stainless steel or alloys thereof, all of which are deformed into a closed position about the vessel and because of the nature of the metal stay deformed and resist any force by the vessel to expand or open up.

Metal clips cause a certain amount of interference with high technology diagnostic modalities, including Computer Tomography (CATSCAN) and Magnetic Resonance Imaging (MRI). In particular, the new and emerging MRI techniques place stringent demands on the non-interference properties of clips. To aggravate the situation even more, recent developments in in vivo Magnetic Resonance Spectroscopy (MRS) create even greater demands on minimizing magnetic field interferences. Some existing metal clips may preclude the use of MRS data taken in the proximity of the metal clips.

To overcome the above problems, in recent years plastic clips have been introduced. The plastic clips now in the market may be biodegradable and absorbable polymeric or other material or nonabsorbent.

Some of the currently available clips may have the disadvantage of providing opportunities for infection in the patient. Infectious organisms may enter the body from a contaminated or infected surgical field or may be already present in the body. Certain ligating clips may allow infectious organisms to grow thereon or may harbor such organisms on the clip or in microcracks formed in the surface of the clip. Therefore, there is a need in the art for a surgical ligating clip which is resistant to harboring infectious organisms or which can kill such organisms existing in the body or in the area of the clip, eliminating the opportunity for infection in the body. Moreover, there is a need in the art for both metal and polymeric ligating clips which are resistant to or which eliminate infectious organisms. This invention provides such a ligating clip which has antimicrobial or antibiotic properties.

### SUMMARY OF THE INVENTION

The present invention provides a ligating clip for use in a patient's body comprising a metal ligating clip having one or more surfaces coated with an antimicrobial or antibiotic coating composition. In an alternative embodiment, the antimicrobial or antibiotic composition is compounded into the metal. In a further embodiment, the invention provides a polymeric or absorbable ligating clip for use in a body comprising a ligating clip formed from a polymer or absorbable material having one or more surfaces coated with an antimicrobial or antibiotic composition. In a further embodiment, the polymeric or absorbable ligating clip is made from a polymer or absorbable material which comprises a polymer or absorbable material and an antimicrobial or antibiotic composition. The patient may be a human or any other animal patient.

In a further embodiment of the invention, there is provided a method for inhibiting the growth of or killing microorganisms comprising coating a ligating clip with an antimicrobial or antibiotic coating composition.

In a still further embodiment of the invention, there is provided a method for inhibiting the growth of or killing microorganisms comprising coating a ligating clip applying instrument with an antimicrobial or antibiotic coating composition.

### BRIEF DESCRIPTION OF THE DRAWINGS

FIG. 1 is a drawing of an example of a metal chevron shaped ligating clip which can be provided according to the present invention.

FIG. 2 is a drawing of an example of a ligating clip which can be provided according to the present invention.

FIG. 3 is a drawing of an example of a ligating clip applying instrument which can be provided according to the present invention.

FIG. 4 is a drawing of an endoscopic ligating clip applier which can be provided according to the present invention.

### DETAILED DESCRIPTION OF THE INVENTION

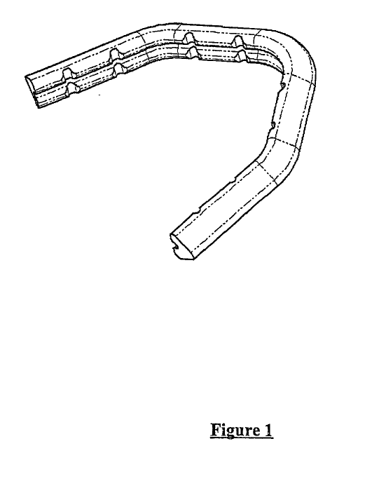

The ligating clip of the present invention may be any ligating clip which can be manufactured to include an antimicrobial or antibiotic coating or component as taught herein. Ligating clips which are useful in the present invention include those disclosed in U.S. Pat. Nos. 4,834,096, 5,062,846 and 5,100,416, each of which is incorporated herein by reference in its entirety. For example, in one embodiment shown in FIG. 1, there may be provided a chevron shaped ligating clip which has an antimicrobial or antibiotic coating thereon or antimicrobial or antibiotic material incorporated therein. In another aspect of the invention, the ligating clip is a polymeric clip including an antimicrobial or antibiotic material having first and second curved leg members joined at their proximal ends by a reduced thickness hinge portion and movable from an open position to a closed position for clamping a vessel between curved opposing inner surfaces which are substantially parallel when the clip is closed. The first leg member has a concave inner surface and a hook portion at its distal end curved toward the second leg member. The hook portion is disposed to engage the outer surface of the end of the second leg member when the clip is in the closed position. The outer surface of the second leg member opposite the inner convex surface is concave in shape. This configuration provides a more secure latching mechanism, since any forces by the clamped vessel tending to open the clip will force the second leg to lengthen and the first leg member to shorten moving the distal end of the second leg member into further engagement with the hook portion. Because the thickness of the second leg member is smaller than it would have been without the concave outer surface, the second leg member will deflect upon clamping or in response to the forces exerted on it by the clamped vessel and because the thickness of each leg between its inner and opposite outer surfaces between the hinge and distal end is substantially equal to the thickness of the other leg, the total deflection necessary to accommodate closing and clamping of the vessel is distributed between the two legs helping to avoid breakage or failure of either leg.

In another aspect of the invention, the ligating clip having one or more surfaces coated with an antimicrobial or antibiotic coating composition or having such material incorporated therein is a surgical ligating clip comprising first and second curved leg members joined at their proximal ends by a resilient hinge means, each leg member having a vessel contacting inner surface and an opposite outer surface, the vessel clamping inner surface being in opposition to the vessel clamping inner surface of the other leg member, the first leg member terminating at its distal end in a deflectable hook member curved toward the second leg member, the second leg member terminating at its distal end is a complimentary locking portion to the hook member whereby when the first and second leg members are moved from an open position to a closed position about the hinge means, the hook member deflects about the distal end of the second leg member to lock the clip in a closed position, the inner surface of the first leg member having a concave radius of curvature between the hinge means and the hook member, the inner surface of the second leg member having a convex radius of curvature between the hinge means and its distal end and the outer surface of the second leg member having a concave radius of curvature between the hinge means and its distal end.

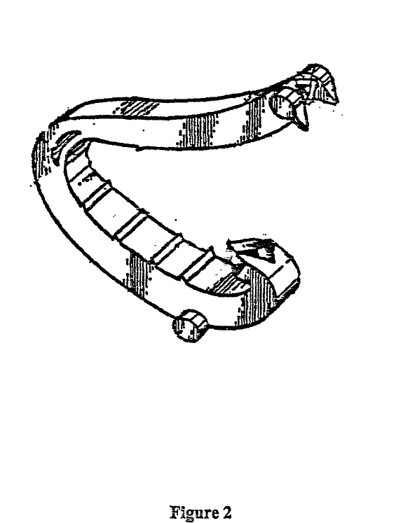

In another aspect of the invention shown in FIG. 2, the ligating clip is of a polymeric material which has one or more surfaces coated with an antimicrobial or antibiotic coating composition or has such material incorporated therein and comprises first and second curved leg members joined at their proximal ends by a hinge means. The first and second curved leg members are disposed to be latched together in the closed position at their distal ends. The leg members each include complementary curved, opposing inner surfaces, the inner surface of the first leg being concave in shape. The first leg member further includes a hook portion joined at its distal end and curved toward the second leg member. The hook portion includes a continuously curved outer surface extending from the outer surface of the distal end of the first leg and a distal tip portion forming a sharp pointed distal tip extending rearwardly toward the proximal end of the first leg. The hook portion may also include a sharp pointed member attached to the outer surface of the distal tip portion. The hook portion is disposed to engage the outer surface of the distal end of the second leg member when the clip is in the closed position. The distal end of the second leg member includes a groove through which the sharp pointed distal tip presses when the first and second leg members are moved from the open position to a closed position. Where the clip includes a sharp pointed member the sharp pointed member passes through the groove ahead of the sharp pointed distal tip. The sharp pointed member engages, stretches and penetrates connective tissue connected to the vessel to be clamped. In the stretched position, the connective tissue is more easily penetrated and cut by the sharp distal tip as the clip is closed.

In another aspect of the invention, the ligating clip is a polymeric surgical clip having an antimicrobial or antibiotic coating or having such material incorporated therein wherein the clip comprises first and second leg members joined at their proximal ends by a resilient hinge means, each leg member having a vessel clamping or contacting inner surface and an opposite outer surface, the vessel clamping or contacting inner surface being in opposition to the vessel clamping or contacting inner surface of the other leg member, the first leg member terminating at its distal end in a deflectable hook member curved toward the second leg member, the second leg member terminating at its distal end in a locking portion complementary to the hook member whereby when the first and second leg members are moved from an open position to a closed position about the hinge means, the hook member deflects about the distal end of the second leg member to lock the clip in a closed position, the hook member having a continuously curved outer surface extending distally from the outer surface of the first leg member, side surfaces and an inner surface; the hook member further comprising a distal tip portion terminating in a sharp pointed distal tip extending rearwardly toward the proximal end of the first leg, the distal end of the second leg member including a groove through which the distal tip passes when the first and second leg members are moved from an open position to a closed position, whereby connective tissue adjacent the vessel to be clamped is cut or stretched, which aids in locking the first and second leg members when the legs are closed.

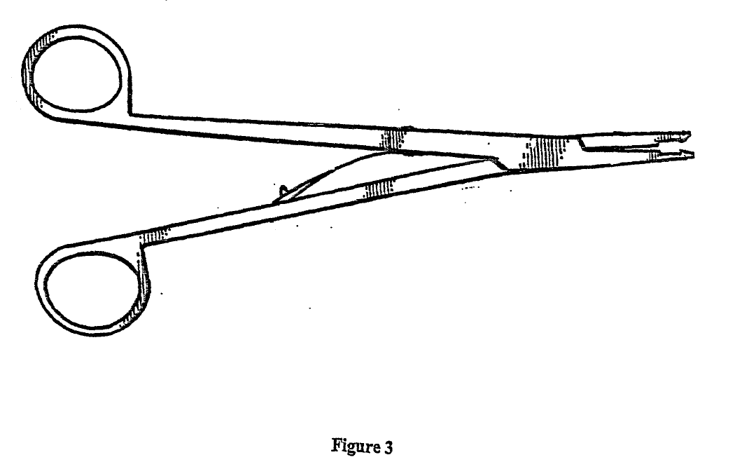

Ligating clip applying instruments are known in the art, such as described in U.S. Pat. No. 5,100,416. In accordance with one aspect of the present invention, such instruments may be coated with antimicrobial and/or antibiotic materials in order to provide resistance to the growth of organisms or to kill such organisms during use of these instruments near or in a patient's body, in storage or in a sterilization area. The patient may be a human patient or any other animal patient in need of the use of a ligating clip. A ligating clip applying instrument for applying a ligating clip generally has a pair of handles pivoted about a hinge point and extends beyond the hinge point to form a pair of clip closing jaws equipped with means for engaging bosses located on the sides of the first and second leg members. Such ligating clip applying instrument is illustrated in FIG. 3.

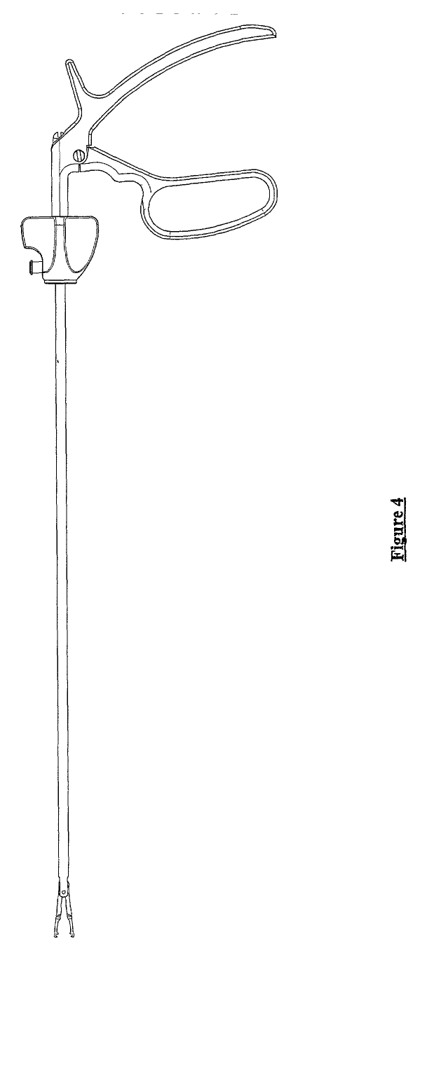

In another aspect of the invention, endoscopic applier devices such as shown in FIG. 4 may be coated with antimicrobial and/or antibiotic materials in order to provide resistance to the growth of organisms or to kill such organisms during use of these instruments.

The ligating clip of the invention may be made of a metal, a polymeric material or a bioabsorbable or biodegradable material. Metal ligating clips are known in the art and may be made from various metals or metal alloys. The most common metals for the manufacture of ligating clips are tantalum, titanium or stainless steel, or alloys of these metals. The most preferred metal for the surgical clips of the present invention is titanium.

Polymeric or plastic ligating clips have also been described, such as in U.S. Pat. Nos. 5,062,846 and 4,834,096, incorporated herein by reference. The polymeric or plastic ligating clip according to the present invention may be of any material meeting the requirements of a ligating clip intended for use in a patient and which can be provided with an antimicrobial or antibiotic material coated thereon or incorporated into the polymer material itself. The ligating clip may be of thermoplastic or thermoset polymers. The ligating clip preferably will be made from one of the engineering plastics commercially available for surgical devices. Such plastics will be biocompatible and include polymers such as polyethylene terephthalate, polybutylene terephthalate, polyacetal, polytetrafluroethylene, high density polyethylene, low density polyethylene, ethylene tetrafluoroethylene and polyoxymethylene. Preferably, the plastic material will be a thermoplastic material that can be injection molded, extruded or otherwise thermally processed into shaped articles.

The ligating clip of the invention in one aspect of the invention is an absorbable or biodegradable clip, typically made from a starch-based material or biodegradable polymer. Such biodegradable polymers are known in the art, for example, homopolymers or copolymers of glycolide, lactide, caprolactone, pdioxanone and trimethylene carbonate. Antimicrobial or antibiotic material may be coated on the absorbable clip or incorporated in the material from which it is made.

As used herein, antimicrobial means any agent which is antagonistic to microbes. Antibiotic refers to any agent which inhibits the growth of other organisms, particularly microorganisms. Some agents will qualify as both an antimicrobial and an antibiotic material.

It may be desirable for the antimicrobial or antibiotic material to be effective against a broad range of infectious agents or pathogens. For example, antimicrobial or antibiotic materials effective against one or more of the following infectious agents may be useful: staphylococcus aureus, coagulase-negative staphylococcus, streptococcus, β-hemolytic, streptococcus pneumoniae, enterococcus, corynebacterim jeikeium, listeria monocytogenes, moraxella catarrhalis, neisseria gonorrhoeae, neisseria meningitidis, citrobacter, enterbacter, escherichia coli, klebsiella pneumoniae, proteus mirabilis, salmonella, serratia, shigella, acinetobacter, aeromonas hydrophila, hemophilus influenzae, legionella pneumophila, pasteurella multocida, pseudomonas aeruginosa, stenotrophomonas maltophilia, clostridium difficile, clostridium, peptostreptoccus, bacteroides fagilis group, prevotella, mycobacterium avium-intracellulare, mycobacterium tuberculosis, chlamydia, mycoplasma pneumoniae, among others.

Antimicrobial or antibiotics which have been found to be effective against one or more of these infectious agents include ampicillin, oxacillin, penicillin G, piperacillin, ticarcillin-carbenicillin, ampicillin/sulbactam, aztreonam, imipenem, meropenem, piperacillin/tazobactam, ticarcillin/clavulanate, cefazolin-cephalothin, cephalexin, cefaclor, cefamandole-cefonicid, cefotetan, cefoxitin-cefinetazole, cefuroxime axetil, cefuroxime sodium, cefdinir, cefixime, cefoperazone, cefotaxime, cefpodoxime, ceftazidime, ceftibuten, ceftizoxime, ceftriaxone, cefepime, ciprolfloxacin, gatifloxacin, levofloxacin, moxifloxacin, amikacin, gentamicin, netilmicin, spectinomycin, streptomycin, tobramycin, azithromycin, clarithromycin, erythromycin, quinupristin/dalfopristin, linezolid, chloramphenicol, clindamycin, fosfomycin, metronidazole, nitrofurantoin, rifampin, sulfonamides, tetracyclines, vancomycin, among others. These agents fall generally into the categories of penicillins, cephalosporins, quinolones, aminoglycosides, macrolides, streptogramin, oxazolidinone and other antimicrobials.

Antimicrobial materials useful in this invention include any antimicrobial materials which can be coated onto or included within the material from which the ligating clip or applier is made. Particularly useful antimicrobial materials include metals known to have antimicrobial properties such as silver, gold, platinum, palladium, iridium, tin, copper, antimony, bismuth, selenium and zinc. Compounds of these metals, alloys containing one or more of these metals, or salts of these metals may be coated onto the surface of the ligating clip or added to the material from which the ligating clip is made during the manufacture of the clip or compounded into the base material. A preferred antimicrobial material will contain silver ions and may be obtained through the use of silver salts, such as silver acetate, silver benzoate, silver carbonate, silver iodate, silver iodide, silver lactate, silver laurate, silver nitrate, silver oxide, silver palmitate, silver protein, or silver sulfadiazine, among others. Other preferred antimicrobial materials are selenium and copper.

Antibiotic materials to be used with the ligating clips of this invention may include any antibiotic capable of being coated onto a ligating clip or applier or incorporated within the material from which the clip is made. Such antibiotics include oxacillin, aminoglycosides, erythromycin, ciprolfloxacin, cephalosporins, quinolones and vancomycin, among others. Antibiotics discovered hereafter may also be used where such antibiotic is capable of incorporation into a coating for or material for manufacture of a ligating clip or applier. In a preferred embodiment, the antibiotic material will be cephalosporin or aminoglycosides.

Antimicrobial and/or antibiotic materials may be chosen based upon the particular application anticipated for the ligating clip. For example, it may be desirable to use a timed release or leachable content material for a particular use. The material comprising the clip may also affect the choice of antimicrobial and/or antibiotic material. For example, metal ligating clips which are to be provided with antimicrobial or antibiotic coatings, preferably, will require antimicrobial and/or antibiotic materials which can be coated onto the metal with satisfactory adhesion so the resistance to harboring infectious organisms, or ability to kill such organisms is present throughout the use of the ligating clip. Alternatively, where the antimicrobial and/or antibiotic material is to be compounded into the metal prior to its formation into a ligating clip, the antimicrobial and/or antibiotic material should be selected so that the material can be readily incorporated into the metal of the clip. Preferably where a metal ligating clip is desired, the metal will be titanium and the antimicrobial will be silver ion.

Likewise, where the ligating clip is of a polymeric material, the antimicrobial and/or antibiotic materials may be selected such that they can be used as or in a coating material or be incorporated into the polymeric material itself. For example, materials such as silver ions, selenium, silver zeolite may be used, or any commercially available additives, e.g., such as Heathshield,® among others.

The antimicrobial and/or antibiotic materials may be coated onto the surface of a ligating clip or ligating clip applying instrument by any method known to those of skill in the art. The coating method will be determined by the material of the clip and the antimicrobial and/or antibiotic material utilized. Such methods include dipping, spraying, rolling, plating and embedding the coating into the surface by any means, among others. For example, polymeric clips and appliers may be coated by dip or spray coating polymeric resin and crosslinker with the antimicrobial or antibiotic material as substituent or dissolved within the polymer. Curing typically is achieved chemically, photochemically or thermally. Other common methods include dip or spray coating water insoluble resin containing antimicrobial components followed by drying or grafting antimicrobials directly onto the substrate chemically or photochemically.

Examples of ways to form the clips and appliers of the invention include blending an antimicrobial agent with a polymer and then forming the polymer into a ligating clip or clip applier. Alternatively, the antimicrobial may be in a solution with the polymer to form a coating. In another aspect of the invention, the antimicrobial may be attached to a polymeric ligating clip or applier by a chemical modification of the surface such as surface grafting by hydrolyzable linkage of the antimicrobial to the surface or by photolinking the antimicrobial to the surface. Surface polymerization, derivatization or absorption may also be used. Other examples of obtaining a surface bound antimicrobial include any existing means, such as ion implantation, chemical modification of the surface, photochemical or chemical grafting or formation of a crosslinked surface immobilized network. Silver ions, where used, may be deposited on the surface of the clip or applier by vacuum deposition, ion sputtering or surface deposition, among others. The surface of the clip may be pretreated according to known methods such as plasma treatment prior to exposure to the coating material. Where solvents are present in the antimicrobial or antibiotic coating composition such solvents must be biocompatible if residue remains after the coating is applied.

Antimicrobial and/or antibiotic materials may be incorporated into the metal or polymeric material of the clip itself. In one embodiment, the antimicrobial and/or antibiotic material is added to the polymer material prior to molding or extruding the final ligating clip. Where it is preferred to incorporate the antimicrobial and/or antibiotic material into the polymer, such inclusion in the polymer may take place during polymerization. In an alternate embodiment, the antimicrobial and/or antibiotic material is compounded into the metal where a metal clip is desired.

One aspect of the invention is directed to a method for inhibiting the growth of microorganisms or killing the microorganisms on a ligating clip comprising coating the ligating clip with an antimicrobial or antibiotic coating composition. Alternatively, the invention provides for inclusion of antimicrobial or antibiotic formulations in the materials used to construct the ligating clip. Likewise, the invention is directed to a method for inhibiting the growth of microorganisms or killing the microorganisms on a ligating clip applying instrument comprising coating the ligating clip applying instrument with antimicrobial or antibiotic substances or including such components within the material used to form the instrument.

By use of the method of the invention, the ligating clips and ligating clip applying instruments so treated resist harboring or kill microorganisms in or around the area of these medical tools. The coatings or antimicrobial and/or antibiotic materials included within the ligating clips and clip applying instruments have been proven to terminate microorganisms in a variety of ways, depending on the type of coating used or the ability of the antimicrobial and/or antibiotic materials to be included within the ligating clip or clip applying instrument. Thus, the present invention may provide the desired antimicrobial or antibiotic effect by a variety of mechanisms, including preventing adherence of an organism to a surface of the clip or applier, providing slow release of an antimicrobial into the surrounding area or immobilizing antimicrobials on the surface of the clip or applier for long term effects. For example, where the coating material contains silver ions, these ions may leach into the area surrounding the clip with the coating and kill microbial organisms present in that area and/or kill microbes that contact the clip surface. This provides a quick but short lived effect. Alternatively, the silver ions may be attached to the surface of the ligating clip and organisms which come in contact with the surface are killed. This method allows a longer lasting effect and keeps organisms off the surface of the ligating clip. Preferably, these methods are combined to kill microbes in the area around the clip or clip applier and on the instrument itself.

In another embodiment, where selenium is used, the selenium is bonded to the surface of the ligating clip, providing a long lasting antimicrobial coating. In yet another embodiment, antibiotic applied to the surface of a ligating clip may leach from the surface to affect any microbial organisms in proximity to the surface.

While the invention has been described in detail and with reference to specific embodiments thereof, it will be apparent to one skilled in the art that various changes and modifications can be made without departing from the spirit and scope thereof.

We claim:

1. A polymeric ligating clip for use in a body having one or more surfaces coated with an antimicrobial or antibiotic coating composition.

2. The ligating clip according to claim 1 wherein the antimicrobial coating composition comprises silver ions.

3. The ligating clip according to claim 1 wherein the antimicrobial coating composition comprises at least one of the compounds selected from the group consisting of selenium, copper, silver acetate, silver benzoate, silver carbonate, silver iodate, silver iodide, silver lactate, silver laurate, silver nitrate, silver oxide, silver palmitate, silver protein, and silver sulfadiazine.

4. The ligating clip of claim 1 wherein the antibiotic coating composition is selected from the group consisting of oxacillin, aminoglycosides, erythromycin, ciprolfloxacin, cephalosporins, quinolones and vancomycin.

5. A metal ligating clip for use in a body having one or more surfaces coated with an antimicrobial or antibiotic coating composition.

6. The ligating clip according to claim 5 wherein the antimicrobial coating composition comprises silver ions.

7. The ligating clip according to claim 5 wherein the antimicrobial coating composition comprises at least one of the compounds selected from the group consisting of selenium, copper, silver acetate, silver benzoate, silver carbonate, silver iodate, silver iodide, silver lactate, silver laurate, silver nitrate, silver oxide, silver palmitate, silver protein, and silver sulfadiazine.

8. The ligating clip of claim 5 wherein the antibiotic coating composition is selected from the group consisting of oxacillin, aminoglycosides, erythromycin, ciprolfloxacin, cephalosporins, quinolones and vancomycin.

9. A ligating clip for use in a body comprising a ligating clip formed from a polymer material wherein the polymer material includes an antimicrobial or antibiotic composition.

10. The ligating clip according to claim 9 wherein the antimicrobial composition comprises at least one of the compounds selected from the group consisting of selenium, copper, silver acetate, silver benzoate, silver carbonate, silver iodate, silver iodide, silver lactate, silver laurate, silver nitrate, silver oxide, silver palmitate, silver protein, and silver sulfadiazine.

11. The ligating clip of claim 9 wherein the antibiotic composition is selected from the group consisting of oxacillin, aminoglycosides, erythromycin, ciprolfloxacin, cephalosporins, quinolones and vancomycin.

12. A method for inhibiting the growth of or killing microorganisms comprising coating a ligating clip or a ligating clip applying instrument with an antimicrobial or antibiotic coating composition.

13. The method according to claim 12 wherein the antimicrobial coating composition comprises at least one of the compounds selected from the group consisting of selenium, copper, silver acetate, silver benzoate, silver carbonate, silver iodate, silver iodide, silver lactate, silver laurate, silver nitrate, silver oxide, silver palmitate, silver protein, and silver sulfadiazine.

14. The method of claim 12 wherein the antibiotic coating composition is selected from the group consisting of oxacillin, aminoglycosides, erythromycin, ciprolfloxacin, cephalosporins, quinolones and vancomycin.

15. The method of claim 12 wherein the ligating clip is formed from a polymer.

16. The method of claim 12 wherein the ligating clip or ligating clip applying instrument is formed from a metal or a metal alloy.

17. The method of claim 12 wherein the ligating clip is formed from an absorbable material.

18. The method of claim 12 wherein the antimicrobial or antibiotic coating composition kills microorganisms by leaching into an area surrounding the ligating clip or ligating clip applying instrument, by contacting the microorganisms on a surface of the ligating clip or ligating clip applying instrument or by a combination thereof.

19. A method for inhibiting the growth of or killing microorganisms comprising incorporating an antimicrobial or antibiotic composition into the composition of a ligating clip or ligating clip applying instrument.

20. The method according to claim 19 wherein the antimicrobial composition comprises at least one of the compounds selected from the group consisting of selenium, copper, silver acetate, silver benzoate, silver carbonate, silver iodate, silver iodide, silver lactate, silver laurate, silver nitrate, silver oxide, silver palmitate, silver protein, and silver sulfadiazine.

21. The method of claim 19 wherein the antibiotic composition is selected from the group consisting of oxacillin, aminoglycosides, erythromycin, ciprolfloxacin, cephalosporins, quinolones and vancomycin.

22. The method of claim 19 wherein the ligating clip is formed from a polymer or an absorbable material.

23. The method of claim 22 wherein the polymer is polyethylene terephthalate, polybutylene terephthalate, polyacetal, polytetrafluoroethylene, high density polyethylene, low density polyethylene, ethylene tetrafluoroethylene or polyoxymethylene.

24. A ligating clip for use in a body comprising a ligating clip formed from a metallic material wherein the metallic material includes an antimicrobial or antibiotic composition.

25. The ligating clip according to claim 24 wherein the antimicrobial composition comprises at least one of the compounds selected from the group consisting of selenium, copper, silver acetate, silver benzoate, silver carbonate, silver iodate, silver iodide, silver lactate, silver laurate, silver nitrate, silver oxide, silver palmitate, silver protein, and silver sulfadiazine.

26. The ligating clip of claim 24 wherein the antibiotic composition is selected from the group consisting of oxacillin, aminoglycosides, erythromycin, ciprolfloxacin, cephalosporins, quinolones and vancomycin.

26. An absorbable ligating clip for use in a body having one or more surfaces coated with an antimicrobial or antibiotic coating composition.
